(19) United States
(12) Patent Application Publication
(10) Pub. No.: US 2020/0069836 A1
(43) Pub. Date: Mar. 5, 2020

(54) POLYMER-BASED ARTERIAL HEMANGIOMA EMBOLIZATION DEVICE, MANUFACTURING METHOD AND APPLICATION OF SAME

(71) Applicant: BEIJING ADVANCED MEDICAL TECHNOLOGIES, LTD. INC., Beijing (CN)

(72) Inventors: Hugh Qinghong Zhao, Pleasanton, CA (US); Qinghua Zhao, Beijing (CN); Dengqiang Jia, Beijing (CN); Shujun Cui, Beijing (CN); Qing Liu, Beijing (CN)

(21) Appl. No.: 16/468,109

(22) PCT Filed: Dec. 13, 2016
(86) PCT No.: PCT/CN2016/109635

§ 371 Date: Jun. 10, 2019

(51) Int. Cl. A61L 24/04; A61L 24/00; A61L 24/08; A61B 17/12; B29C 48/00; B29C 53/80; B29C 48/92; B29C 48/05

(52) U.S. Cl. CPC A61L 24/046; A61B 2017/00004; A61L 24/001; A61L 24/043; A61L 24/08; A61L 24/0015; A61B 17/1215; B29C 48/0019; B29C 53/80; B29C 48/92; B29C 48/05; B29C 48/0011; A61L 2300/44; A61L 2420/02; A61B 17/12113; A61B 17/12109; A61L 24/0042

(57) ABSTRACT

A polymer-based embolization device comprises a helix constructed by a linear structure. The linear structure is either a fibrous structure or composed of an A structure (1) and a B structure (2), wherein the A structure (1) is a protrusion on the linear structure and the B structure (2) is a pillar-shaped structure positioned between two A structures (1) for connecting the two A structures (1). The embolization device adopts a linear structural design and is integrally manufactured using a polymer material via a four-axis rapid forming system or via a compression method, thereby addressing issues of generation of image artifacts during CT and magnetic resonance imaging. The combination of design, material, and technique of the invention provides the device with improved flexibility and embolus formation, and can satisfy different clinical requirements. When a biodegradable macromolecular material is selected for manufacturing, blood vessel obstruction caused by implant degrading can be avoided, allowing the blood vessel to return to a normal structural state.

### TECHNICAL FIELD OF THE INVENTION

The invention relates to a polymer-based arterial embolization device, and a manufacturing method and use thereof.

### BACKGROUND OF THE INVENTION

Aneurysm is a disease caused by the structural and hemodynamic changes of blood vessel wall due to a variety of factors. The wall of the aneurysm becomes thin, and once it is ruptured, the patient is at risk of life due to massive loss of blood in a short period of time.

The previous treatment was performed mainly through surgical clipping. For sites or some other medical conditions where direct access for surgery is difficult, the operator had to use intravascular embolization for interventional treatment. In 1974, Serbineko firstly used a detachable balloon to embolize intracranial aneurysms. At present, the use of coils for embolization treatment has been popularized, wherein a number of coils are implemented into aneurysm-bearing blood vessels so as to adsorb blood components to agglutinate and form embolization in the aneurysm. The purpose of treatment is achieved by isolating the aneurysm from the blood circulation of aneurysm-bearing artery.

The coils currently available in the market are mainly bare metal coils and biologically modified coils. Representative commercial products include: Flipper, Nester, MReye and Embolization Coils of COOK, which are made of nickel-chromium alloy, platinum-tungsten alloy, chromium-nickel-iron alloy, 304 stainless steel wire, platinum and embedded nylon 66 fiber; Interlock-35 and Fibered IDC of Boston Scientific, which are made of platinum-tungsten alloy and contain polyester fiber; Jasper of Achieva Medical, which is made of platinum-tungsten alloy; MicroPlex of MicroVention, which is made of platinum-tungsten alloy and has anti-stripping filaments made of polyolefin elastomer; Nit-Occlud of Pfm Medical, which is made of ferro-nickel alloy; Axium GLA (made of platinum-tungsten alloy, containing polypropylene anti-spin wire, and having PGLA ciliated wire on the coil), Axium Nylon (made of platinum-tungsten alloy, with nylon ciliated wire and polypropylene core wire on the coil), Axium (composed of platinum-tungsten alloy wound wire, polypropylene core wire and 316L stainless steel release zone), and NXT Detachable Coils (made of platinum-iridium alloy, coated with parylene polymer, and having a core wire structure with a material of nickel-titanium alloy) of MicroTherapeutics; GDC (made of platinum-tungsten alloy with filamentary material (polypropylene) in the center of some types of coils to help resist unwinding) and Matrix (made of platinum-tungsten alloy, coated with an absorbable polymer (polyglycolide and polylactide (90/10) polymer)) of Stryker Neurovascular; Codeman of Medos International SARL (made of platinum-tungsten alloy, with polyglycolic acid resistant wire inside the coil).

A coil packing rate (coil volume/aneurysm volume) of 25% is a compact packing, as described in the article by Kadirvel R, Ding Y H, Dal D, et al. (Proteomic analysis of aneurysm healingmechanism after coil embolization: Comparison of dense packing with loosepacking [J]. AJNR Am Neuroradiol, 2012, 33(6): 1177-1181). Before a stable embolization is formed a slow blood flow can permeate, so rapid organization in the aneurysm cavity and rapid endothelialization at the aneurysm neck are critical, it is necessary that the implant rapidly embolizes without causing an inflammatory reaction.

Organization is a process in which foreign matter in a tissue formed due to inflammation or injury is treated by dissolution, absorption, or the like. In the process, granulation tissue is formed, phagocytes take up foreign matters, the foreign matters are dissolved and absorbed because of the action of enzymes, and then the disease is cured along with scarring of the granulation tissue.

CN 1899223A discloses a biocoated spring coil, wherein the coating is composed of a biodegradable polymer or hydrogel liquid or a drug capable of efficiently forming an embolization. The coil coated with the polymer can accelerate the formation of clot tissue in aneurysm and can more effectively ensure the embolism of aneurysm; the hydrogel has a self-expanding capability in blood or humid environment and can effectively deal with the problem of wide neck or huge aneurysm.

The bio-modified coil is the tendency of coil products in the future because of its favorable characteristics of facilitating thrombosis and organization. However, the simple bio-modification cannot improve the long-term mass effect of the metal. The density of metal materials is relatively high compared to the tissue of a human body, which may compress peripheral blood vessels and nerves in the long run. Once the aneurysms are recanalized or the arteries are ruptured, irreparable results may be caused. In addition, because of the large difference in physical and mechanical properties between the metal materials and the tissue, the metal materials are not easy to be randomly attached to the wall for shaping. Meanwhile, as for larger aneurysms, it is often necessary to pack multiple coils. Metal coils will produce strong metal artifacts in CT and magnetic resonance imaging (MRI), greatly affecting observation of the surrounding tissue, which can seriously affect subsequent CT and MRI examination of the patient.

In order to solve these problems, research work on degradable coils has been carried out subsequently. CN 104739478A discloses a coil comprising a first coil and a second coil, wherein one of the coils is made of degradable material and the other coil is made of radiopaque material; CN 104398283A discloses a coil with an expandable degradable polymer which can be selectively and slowly decomposed after the lesion endothelialisation process is completed so as to reduce the mass effect of the device. However, these techniques can only partially degrade the implant and the hidden danger still exists.

To this end, CN 105411643A discloses a coil made of magnesium or a magnesium alloy material, which can completely degrade after implantation, but the disadvantages are: magnesium metal is degraded too quickly to fill an aneurysm with collagen and myofibroblasts; the intratumoral embolization is unstable; an aneurysm recurrence is likely to happen. In addition, the problem that artifacts are generated during CT and magnetic resonance imaging (MRI) cannot be avoided.

### SUMMARY OF THE INVENTION

To address the defects and clinical requirements in the prior art, the present invention has developed a polymer-based embolization device by selecting a polymer raw material, adopting a four-axis rapid forming process or a compression molding method, and using a special structural design, which has a better embolizing effect, can be used for embolization treatment of vascular malformations, and can avoid metal artifacts generated during CT or MRI imaging.

The present invention provides a polymer-based embolization device, which is a helix constructed by a linear structure; wherein the linear structure is either a fibrous structure or composed of an A structure and a B structure, wherein the A structure is a protrusion on the linear structure and the B structure is a pillar-shaped structure positioned between two A structures for connecting the two A structures.

The embolization device according to the present invention, wherein a diameter (D) of the helix may be 1-40 mm, preferably 3-30 mm.

The embolization device according to the present invention, wherein the A structure and B structure may have a plurality of arrangements and the number of A structures between each two B structures may be the same or different. In a preferred embodiment of the present invention, the number of A structures between each two B structures is 1 to 3, more preferably being 1. For example, the arrangement of the A structure and the B structure may be an alternating arrangement like ABABAB, an arrangement like AABAABAAB, an arrangement like AAABAAABAAAB, or a random arrangement like AABABAAAB, etc.

The embolization device according to the present invention, wherein the A structure may be in a variety of shapes, such as spherical, cylindrical, square, cuboid, conical and/or other irregular shapes, preferably spherical or spheroid-like.

In a preferred embodiment, the linear structure of the embolization device of the present invention is schematically illustrated in FIG. 1, wherein a primary structure is a linear structure composed of an A structure and a B structure, and a secondary structure is a helix of the linear structure. As shown in FIG. 1, the A structure may be spherical, cylindrical, square, tapered and the like, and may be circular, elliptical, rectangular, triangular, and other irregular patterns in cross-section. In the present invention, the primary structure refers to an arrangement of an A structure and a B structure, the secondary structure refers to a helix structure of the primary structure as shown in the drawings, i.e., FIG. 2. In FIG. 2, letter D represents the diameter of the helix coil. Furthermore, a tertiary structure refers to a structure in which the secondary structure randomly spirals and stacks, as shown in FIG. 4, that is, a clump or globular structure in which the secondary structure is randomly wound.

In a linear structure according to one embodiment of the present invention, the A structure is a hard segment, the B structure is a soft segment; a certain spatial structure can be formed, and meanwhile, the structure has a certain flexibility, so that the spatial structure can be randomly rotated, compressed, stacked, expanded and attached to a wall in a limited space, generating a flexible final form (i.e., the tertiary structure, as shown in FIG. 4) according to the capacity of a space, thereby achieving adequate packing of the aneurysm, and rapid and efficient embolization during use.

Further, in some embodiments of the present invention, the linear structure may be a fibrous linear structure, i.e., a polymer fiber. As for the embolization device according to the present invention, wherein the A and B structures may be regular or irregular, to which the present invention is not particularly limited. Preferably, the cross section of the A structure can be circular, elliptical, rectangular and/or triangular and the like; the cross-section of the B structure may be circular, elliptical and/or oval, etc.

The embolization device according to the present invention, wherein a size of the fibrous linear structure or sizes of the A structure and the B structure in the embolization device can be designed according to a size of an arterial blood vessel to be embolized. In the case where the linear structure is fibrous, an average diameter of the fibrous linear structure may be 0.05-6 mm. In the case where the linear structure is composed of an A structure and a B structure, an average diameter or length of the cross section of the A structure may be 0.05-6 mm, the average diameter of the cross section of the B structure may be 0.05-0.6 mm, and the length of the B structure may be 0.05-6 mm. Preferably, the average diameter or length of the cross section of the A structure is greater than or equal to the average diameter of the cross section of the B structure. The average diameter of the fibrous linear structure of the present invention as well as the dimensions of the A and B structures may also vary according to clinical requirements. In the linear structure of the embolization device of the present invention, lengths of multiple B structures may be the same or different from each other, and sizes of multiple A structures may also be the same or different from each other.

The embolization device according to the present invention, wherein the linear structure is made from a thermoplastic polymer raw material including a non-degradable thermoplastic polymer and a biodegradable thermoplastic polymer. Preferably, the biodegradable thermoplastic polymer is selected from polylactic acid (PLA) (including L-polylactic acid (PLLA) and D-polylactic acid (PDLA)), polyethylene glycol-polyglycolic acid (PGA), polycaprolactone (PCL), polyethylene glycol (PEG), polyanhydrides, polyhydroxyalkanoates (PHA), polydioxanone, polyiminocarbonates, polyfumaric acid, and copolymers or mixtures thereof; preferably, the non-degradable thermoplastic polymer includes polyethylene terephthalate, nylon, polypropylene, polyethylene, polyurethane, and copolymers or mixtures thereof.

In order to improve the visibility of the device under X-rays, the raw material may also include radiopaque additives. Preferably, the radiopaque additive is one or more selected from the group consisting of: calcium phosphate, metal or metal oxide particles, and iodine compounds, barium sulfate, zirconium dioxide as well as strontium halide used as contrast agents, and the like. In order to improve the visibility of the device under X-rays, metallic development marks may also be introduced on different parts of the embolization device.

The embolization device according to the present invention, wherein the surface or a part of the surface of the embolization device may be treated biologically, chemically, physically or in a combined manner to promote coagulation. Preferably, coagulation may be facilitated by wrapping degradable polymeric cilia around the surface of the embolization device.

Preferably, the surface of the embolization device may be modified to promote coagulation using gelatin, collagen, chitosan, alginate, and the like, as well as the above materials containing an embolizing agent. The gelatin, collagen, chitosan, alginate and the like as well as the materials containing an embolizing agent can be loaded on the embolization device through spraying, dipping and electrostatic spinning.

In another aspect, the present invention also provides a method for manufacturing a polymer-based embolization device of the present invention, the method being carried out using a four-axis rapid forming system as a manufacturingequipment, wherein the four-axis rapid forming system comprises:

(i) a base;

(ii) a three-axis X-Y-Z positioning system connected to the base, the X-Y-Z positioning system defining X, Y, and Z directions, respectively;

(iii) a dispensing system mounted on the X-Y-Z positioning system and movable by means of the X-Y-Z positioning system, the dispensing system comprising an extrusion head;

(iv) a fourth-axis system connected to the base, located below the extrusion head and including a rotating rod connected to the base, wherein the rotating rod is rotatable in a forward or reverse direction about its central axis and the central axis of the rotating rod is parallel to the Y axis; and

(v) a computer control system which can precisely control the X-Y-Z positioning system according to a set program so as to precisely control the movement of the extrusion head of the dispensing system in the X, Y, Z directions and precisely control the rotation of the rotating rod of the fourth-axis system about the central axis thereof; and

the method comprises the steps of:

1) preparing a mold in accordance with the structure of the embolization device to be manufactured;

2) designing a program for a pattern of depositing raw materials for preparing the embolization device by using a computer;

3) fixing the mold at the rotating rod of the fourth-axis system of the four-axis rapid forming system, so that the mold can rotate forwards or backwards along with the rotating rod of the fourth-axis system under the control of a computer control system; and adding raw materials for preparing the embolization device into the dispensing system; and

4) controlling the X-Y-Z positioning system and the fourth-axis system by means of a computer control system according to the program designed in step 2), enabling the dispensing system to accurately extrude raw materials according to a pre-designed deposition pattern so that the extruded raw materials deposit at a specific position of a rotatable mold on the fourth axis or deposit directly on the rotating rod, thereby obtaining the embolization device of the invention.

Preferably, the shape of the mold in step 1) is in a cylindrical shape with a smooth surface (the polymer filaments are deposited directly on the cylindrical surface) or a cylindrical shape with grooves on the surface (the polymer filaments are deposited in the grooves, and the cross section of the grooves may be conical, circular or other shapes). Preferably, the mold is prepared using 3D printing techniques or conventional techniques such as CNC machining.

Preferably, in step 3), the mold is fixed using a clamp or by fitting a hollow mold over the rotating rod of the fourth-axis system.

Preferably, in step 3), the fixing is conducted by replacing the rotating rod of the fourth-axis system with the mold to receive the polymer, fixing the mold to the fourth-axis system and allowing it to rotate in the forward or reverse direction under the control of a computer control system.

The preparation method of the invention utilizes the four-axis rapid forming system in the disclosed patent applications CN 102149859 A and CN 104274867 A entitled to the present applicant. On this basis, further improvements are made according to the characteristics of the embolization device to be prepared. The extruded polymer fibers are deposited onto the mold or directly onto the rotating rod at a set speed, pattern, and in a set wire travel manner.

The linear structural pattern of the embolization device of the present invention is designed using a computer program. The sizes of the fibrous linear structure as well as the A and B structures may be designed by a computer program, and controlled by the rapid forming system, or controlled by both.

In most cases, factors such as the size and geometry of the polymer fibers used in embolization devices, the number of fibers per unit volume, and the structural pattern of the fibers are mostly controlled by certain aspects of the manufacturing equipment, such as by the rotating rod, the mold, or the extrusion head.

The diameter of the mold may be designed according to the required unit helix size of the embolization device. Generally, the diameter of the extruded polymer fibers is determined by the inner diameter of the extrusion head, the extrusion speed, the movement speed of the extrusion head along the rotating rod, and the speed of rotation of the rotating rod. Sometimes, the diameter of the extruded polymer fibers can also be controlled by programming, for example, designed to be repeatedly threading at certain locations to form different or identical cross-sectional diameters of B structures and/or cross-sectional diameters of A structures at different locations.

In another aspect, the present invention also provides another method for manufacturing the polymer-based embolization device of the present invention by compression molding or by injection molding.

In a particular embodiment, when the linear structure is fibrous, the compression molding process may include: melt extruding a polymer pellet through an extrusion device into a polymer filament with a diameter of 0.05-6 mm, spirally winding the polymer filament on a rod-shaped support, and carrying out heat treatment to fix the shape to obtain the embolization device. When the linear structure is composed of an A structure and a B structure, the compression molding method may include: firstly melt extruding a polymer pellet through an extrusion device into a polymer filament with a diameter of 0.05-6 mm, putting the polymer filament into a mold (the mold has an inner cavity with a required arrangement of A structure and B structure) at a molding temperature, then carrying out closed mold pressurization to shape and solidify the polymer filament, spirally winding the polymer filament on a rod-shaped support, and carrying out heat treatment to fix the shape to obtain a polymer helix having the desired arrangement of structures A and B, i.e., the embolization device of the present invention is made.

In addition, the molding method may be any other method that can obtain a polymer helix having a linear structure, thereby manufacturing the embolization device of the present invention.

The embolization device of the present invention may be placed at a desired location by interventional means. Firstly, the device is compressed in a conveying sheath in the form of a linear silk chain, and then reaches the focus, being pushed out, spiraling according to the original pattern, and packing the focus cavity.

In another aspect, the invention provides the use of the polymer-based embolization device of the invention in the embolization treatment of vascular malformation.

A schematic view of a polymer-based embolization device of the present invention in use is shown in FIG. 3, where 1 is the A structure, 2 is the B structure, and 3 is a delivery instrument.

Preferably, the polymer-based embolization device can be used for embolization treatment of intracranial aneurysms and other vascular malformations, such as arteriovenous malformations and arteriovenous fistulas of the neurovasculature, and for embolization treatment of arteries and veins of the peripheral vasculature, thereby blocking blood flow to the aneurysm or other locations of vascular malformation, forming a embolization and gradually organizing, and the embolization gradually shrinks and eventually disappears as the material degrades. The vascular wall finally restores to the normal shape and functional state.

According to the present invention, a polymer-based embolization device with a linear helix structure is manufactured by using a high-molecular raw material and a four-axis rapid forming system. The polymer-based embolization device and the manufacturing method thereof have the following advantages:

1. Because of the helix linear structure design, the characteristics of rigidity and flexibility are realized, further in line with the expected use of the product.

2. By using the high-molecular material, the problem of artifacts generated during CT and magnetic resonance imaging (MRI) is solved.

3. In case of using the degradable high polymer, the embolization device relieves the permanent threat of the foreign body to the patient, and the blood vessel wall can restore to a natural physiological structure and functional state.

4. Use of the four-axis rapid forming process renders a wider range for selection of materials, instruments with different degradation timing can be manufactured, and compared with the existing embolization device manufacturing process (including welding, laser cutting and weaving technologies), the method is simple, efficient, cost-saving and more flexible.

Because of the perfect combination of the design, the material and the process, the manufactured product can not only attach to the wall randomly, but also form in a supportive manner, being resistant to the scouring and compressing of blood flow; meanwhile, the product can quickly embolize and organize, with a better embolizing effect.

The invention adopts a linear helix design, manufactures the embolization device integrally using a polymer raw material through a four-axis rapid forming system, and solves the problem of artifacts generated during CT and magnetic resonance imaging (MRI). The combination of design, materials and processes renders a device with greater flexibility and embolus formation ability, meeting different clinical needs. When the biodegradable polymer is adopted, the implant can be completely degraded, the occlusion of the implant to the blood vessel is relieved, and the blood vessel restores to a normal structural shape. The manufacturing method is simple and rapid to operate, easy to change, low in cost and suitable for industrialization.

### BRIEF DESCRIPTION OF THE DRAWINGS

For a more complete understanding of the features and advantages of the present invention, reference is now made to the detailed description of the invention along with the accompanying figures and in which:

FIG. 1 is a schematic view showing a primary structure of the embolization device of the present invention;

FIG. 2 is a schematic view showing a secondary structure of the embolization device of the present invention;

FIG. 3 is a schematic view showing the polymer-based embolization device of the present invention in use;

FIG. 4 is a photograph of the final configuration of the embolization device made in Example 1.

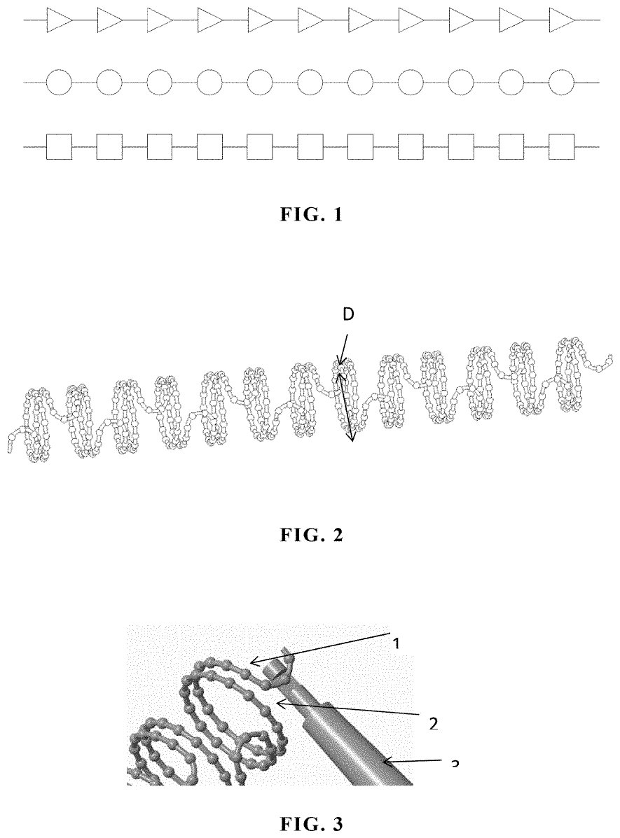

### DETAILED DESCRIPTION OF THE INVENTION

The invention is further illustrated by the following examples, which are intended to be illustrative only and are in no way intended to limit the scope of the invention.

### Example 1

The example provides a polymer-based embolization device used in peripheral embolization surgery and a manufacturing method thereof, the method comprising:

1) preparing a mold according to a structure of the embolization device to be manufactured;

2) designing a program for preparing a raw material deposition pattern of the embolization device by using a computer;

3) fixing the mold at the rotating rod of the fourth-axis system of the four-axis rapid forming system, so that the mold can rotate forwards or backwards along with the rotating rod of the fourth-axis system under the control of a computer control system; adding raw materials for manufacturing the embolization device into the dispensing system; and

4) controlling the X-Y-Z positioning system and the fourth-axis system by means of a computer control system according to the program designed in the step 2), enabling the dispensing system to accurately extrude raw materials according to a pre-designed deposition pattern so that the extruded raw materials deposit at a specific position of a rotatable mold on the fourth axis or deposit directly on the rotating rod, thereby finishing the manufacturing of the main body of the embolization device of the invention; and placing in 5 mg/ml collagen type I solution for 1 minute, taking out and washing the surface for 2 to 3 times with PBS solution and then drying in a vacuum oven.

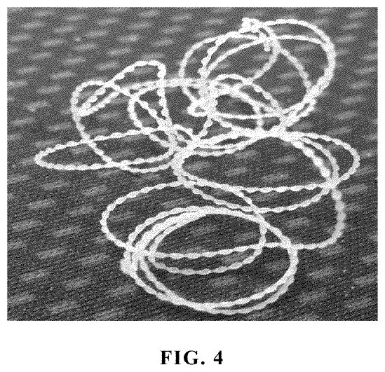

A photograph of the embolization device manufactured in this example is shown in FIG. 4. The embolization device released itself in a free space as spherical, and released in a limited space according to the spatial pattern to form randomly; the raw material used was a mixture of polyethylene terephthalate and contrast agent powder; and the effect of the type I collagen was mainly to modify the surface of the material so as to promote thrombosis in tumors and endothelialization at tumor mouths. The embolization device manufactured in this example has a spherical A structure with a cross-sectional diameter of 0.9 mm, and a B structure with a cross-sectional diameter of 0.5 mm and a length of 0.5 mm.

The device may be used to block blood flow in the peripheral vasculature during embolization procedures and may be delivered through a 5 F catheter.

### Example 2

The example provides a degradable polymer-based embolization device used in vascular embolization surgery and a preparation method thereof, the method comprising:

1) preparing a mold according to a structure of the embolization device to be manufactured;

2) designing a program for preparing a raw material deposition pattern of the embolization device by using a computer;

3) fixing the mold at the rotating rod of the fourth-axis system of the four-axis rapid forming system, so that the mold can rotate forwards or backwards along with the rotating rod of the fourth-axis system under the control of a computer control system; adding raw materials for manufacturing the embolization device into the dispensing system; and

4) controlling the X-Y-Z positioning system and the fourth-axis system by means of a computer control system according to the program designed in the step 2), enabling the dispensing system to accurately extrude raw materials according to a pre-designed deposition pattern so that the extruded raw materials deposit at a specific position of a rotatable mold on the fourth axis or deposit directly on the rotating rod, thereby finishing the manufacturing of the main body of the embolization device of the invention; and then manually weaving micro-cilia (diameter 10 μm or so) on the device surface, where the micro-cilia were made by stretching monofilament fibers.

The embolization device released itself in a free space as spherical, and released in a limited space according to the spatial pattern to form randomly; the raw material used was a mixture of PCL and contrast agent powder. The micro-cilia were made by stretching PCL fibers and wound on the surface of the device to improve the roughness of the surface, so that blood coagulation could be promoted, and thrombosis could be more easily induced. The embolization device manufactured in this example has a spherical A structure with a cross-sectional diameter of 0.25 mm, and a B structure with a cross-sectional diameter of 0.15 mm and a length of 0.15 mm.

The device may be used to block blood flow in the vasculature during embolization procedures and may be delivered through a 2 F catheter.

### Example 3

The example provides a polymer-based embolization device used in vascular embolization surgery and a preparation method thereof, the method comprising:

1) preparing a mold with a bead-like groove;

2) preparing polymer fiber filaments with the diameter of 0.2 mm by adopting a conventional hot melting extrusion technology;

3) treating the polymer fiber filaments with an iopamidol solution to obtain polymer fiber filaments containing iopamidol; and

3) placing the polymer fiber filament containing iopamidol into the mold at a forming temperature, closing the mold and pressurizing to form and solidify the polymer fiber filament, removing the polymer fiber filament from the mold to obtain the polymer fiber filament with a bead-like structure, spirally winding the bead-like polymer fiber filament on a rod-shaped support, and carrying out heat treatment to fix the shape to obtain a polymer helix with the bead-like structure; and then manually weaving micro-cilia (diameter 10 μm or so) on the device surface, where the micro-cilia were made by stretching monofilament fibers.

The embolization device released itself in a free space as a helix coil, and released in a limited space according to the spatial pattern to form randomly; and the used polymer was PCL or polyethylene terephthalate. The micro-cilia were made by stretching polymer fibers and wound on the surface of the device to increase the roughness of the surface, so that blood coagulation could be promoted, and thrombosis could be more easily induced. The embolization device manufactured in this example has a spherical A structure with a cross-sectional diameter of 0.2 mm, and a B structure with a cross-sectional diameter of 0.1 mm and a length of 0.1 mm.

The device may be used to block blood flow in the vasculature during embolization procedures and may be delivered through a 2 F catheter.

1. A polymer-based embolization device, the embolization device being a helix constructed by a linear structure, wherein the linear structure is either a fibrous structure or composed of a structure A and a structure B, the structure A is a protrusion on the linear structure and the structure B is a pillar-shaped structure positioned between two A structures for connecting the two A structures.

2. The embolization device according to claim 1, wherein the diameter (D) of the helix is 1-40 mm, preferably 3-30 mm.

3. The embolization device according to claim 1, wherein the A structure is spherical, cylindrical, square, cuboid and/or conical, preferably spherical; preferably, the cross section of the structure A is circular, elliptical, rectangular and/or triangular; and the cross section of the structure B is circular, elliptical and/or oval.

4. The embolization device according to claim 1, wherein an average diameter or length of the cross-section of the A structure is 0.05-6 mm, an average diameter of the cross-section of the B structure is 0.05-6 mm, and a length of the connecting axis is 0.05-6 mm.

5. The embolization device according to claim 1, wherein the embolization device is made of a raw material comprising a biodegradable thermoplastic polymer and/or a non-degradable thermoplastic polymer.

6. The embolization device according to claim 5, wherein the biodegradable thermoplastic polymer is selected from the group consisting of polylactic acid, polyethylene glycol-polyglycolic acid, polycaprolactone, polyethylene glycol, polyanhydrides, polyhydroxyalkanoates, polydioxanone, polyiminocarbonates, polyfumaric acid, and copolymers or mixtures thereof; the non-degradable thermoplastic polymers comprise polyethylene terephthalate, nylon, polypropylene, polyethylene, polyurethane, and copolymers or mixtures thereof.

7. The embolization device according to claim 5, wherein the raw material further comprises a radiopaque additive; preferably, the radiopaque additive is one or more selected from the group consisting of calcium phosphate as well as iodine compound, barium sulfate, zirconium dioxide and strontium halide used as contrast agents.

8. The embolization device according to claim 1, wherein the surface or a part of the surface of the embolization device is wrapped with polymer cilia.

9. The embolization device according to claim 1, wherein the surface of the embolization device is modified with gelatin, collagen, chitosan, alginate or a material containing an embolizing agent.

10. A method for manufacturing a polymer-based embolization device of claim 1, the method being carried out using a four-axis rapid forming system as a manufacturing equipment, wherein the four-axis rapid forming system comprises:
(i) a base;
(ii) a three-axis X-Y-Z positioning system connected to the base, the X-Y-Z positioning system defining X, Y, and Z directions, respectively;
(iii) a dispensing system mounted on the X-Y-Z positioning system and movable by the X-Y-Z positioning system, the dispensing system comprising an extrusion head;
(iv) a fourth-axis system connected to the base, located below the extrusion head and including a rotating rod connected to the base, wherein the rotating rod is rotatable in a clockwise or an anti-clockwise direction around its central axis and the central axis of the rotating rod is parallel to the Y axis; and
(v) a computer controlled system which can precisely control the X-Y-Z positioning system according to a set program so as to precisely control the movement of the extrusion head of the dispensing system in the X, Y, Z directions and precisely control the rotation of the rotating rod of the fourth-axis system around the central axis thereof; and
the method comprises the steps of:
1) preparing a mold or a rotation rod which has a specific surface contour in accordance with the structure of the embolization device to be manufactured;
2) designing a computer program for a pattern of depositing raw materials for preparing the embolization device;
3) g attach the mold to the rotating fourth-axis of the four-axis rapid forming system so that the mold can rotate clockwise or anti-clockwise along with the rotation of the fourth-axis of the four-axis system under the control of a computer control system; and adding raw materials for preparing the embolization device into the dispensing system; and
4) controlling the X-Y-Z positioning system and the fourth-axis system by a computer controlled system according to the computer program designed in step 2), enabling the dispensing system to accurately extrude raw materials according to a pre-designed deposition pattern so that the extruded raw materials deposit onto a specific position of a rotatable mold on the fourth axis or deposit directly on the rotating rod, thereby obtaining the embolization device.

11. The method according to claim 10, the method for making the polymer-based embolization device can be a extrusion or an compress molding.

12. The method according to claim 11, wherein when the linear structure is fibrous, the extrusion molding process includes: melt extruding a polymer through an extrusion device into a polymer filament with a diameter of 0.05-6 mm, spirally winding the polymer filament onto a rod-shaped support, and carrying out heat treatment to fix the shape to obtain the embolization device; when the linear structure is composed of both a structure A and a structure B, the extrusion molding method includes: firstly melt extruding a polymer through an extrusion device into a polymer filament with a diameter of 0.05-6 mm, putting the polymer filament into a mold having an inner cavity with a required arrangement of structure A and B structure at a molding temperature, then carrying out closed mold pressurization to shape and solidify the polymer filament, spirally winding the polymer filament on a rod-shaped support, and carrying out heat treatment to fix the shape to obtain a polymer helix having the desired arrangement of structures A and B, i.e., the embolization device.

13. (canceled)

14. A polymer-based embolization device, the embolization device being a helix constructed by a linear structure, wherein the linear structure is either a fibrous structure or composed of both a structure A and a structure B, the structure A is a protrusion on the linear structure and the structure B is a pillar-shaped structure positioned between two structures A for connecting the two structures A, made by the method comprising:
1) preparing a mold in accordance with the structure of the embolization device to be manufactured;
2) designing a program for a pattern of depositing raw materials for preparing the embolization device by using a computer;
3) attaching the mold to the rotating rod of the fourth-axis system of the four-axis rapid forming system so that the mold can rotate clockwise and anti-clockwise along with the rotating rod of the fourth-axis system under the control of a computer control system; and adding raw materials for preparing the embolization device into the dispensing system; and
4) controlling the X-Y-Z positioning system and the fourth-axis with a computer control system according to the program designed in step 2), enabling the dispensing system to accurately extrude raw materials according to a pre-designed deposition pattern so that the extruded raw materials deposit at a specific position of a rotatable mold on the fourth axis or deposit directly onto the rotating rod, thereby obtaining the embolization device.
